(19) United States
(12) Patent Application Publication
(10) Pub. No.: US 2024/0226124 A9
(43) Pub. Date: Jul. 11, 2024

(54) CANNABIDIOL FOR TREATING NEURODEGENERATIVE DISEASES

(71) Applicant: AUGUSTA UNIVERSITY RESEARCH INSTITUTE, INC., Augusta, GA (US)

(72) Inventors: Babak BABAN, Augusta, GA (US); Jack YU, Augusta, GA (US); Krishnan M. DHANDAPANI, Augusta, GA (US)

(73) Assignee: AUGUSTA UNIVERSITY RESEARCH INSTITUTE, INC., Augusta, GA (US)

(21) Appl. No.: 18/546,720

(22) PCT Filed: Feb. 16, 2022
(86) PCT No.: PCT/US2022/016622

§ 371 Date: Aug. 16, 2023

Related U.S. Application Data
(60) Provisional application No. 63/149,954, filed on Feb. 16, 2021.

(51) Int. Cl. A61K 31/00; A61P 25/28

(52) U.S. Cl. CPC A61K 31/658; A61P 25/28

(57) ABSTRACT

A method of reducing symptoms of a neurodegenerative disease comprising administering an effective amount of cannabidiol to a subject in need thereof to improved cognitive function and ameliorated the pathophysiology of the neurodegenerative disease.

### CROSS-REFERENCE TO RELATED APPLICATIONS

This application claims benefit of and priority to U.S. Provisional Patent Application 63/149,954 filed on Feb. 16, 2021, and where permitted all of which are incorporated by reference in their entireties.

### STATEMENT REGARDING FEDERALLY SPONSORED RESEARCH OR DEVELOPMENT

This invention was made with Government support under NS110378 awarded by the National Institutes of Health. The Government has certain rights in the invention.

### FIELD OF THE INVENTION

The invention is generally directed to methods and compositions for treating neurodegenerative diseases.

### BACKGROUND OF THE INVENTION

Neurological diseases are generally characterized by neuronal loss in one or more regions of the central nervous system. Examples of neurological diseases include Alzheimer's disease, neurofibromatosis, Huntington's disease, depression, amyotrophic lateral sclerosis, multiple sclerosis, stroke, Parkinson's disease and multiple infarction dementia. These diseases are complex in both origin and progression and have proven to be some of the most difficult disease types to treat. In fact, there are no effective drugs that provide substantial therapeutic benefit for some neurological diseases. Given the adverse effects these diseases have on victims, the difficulty of providing treatment is even more tragic.

Alzheimer's disease (AD) is a complex pathological condition associated with irreversible cognitive decline (Breijyeh, Z., et al., Molecules., December 8; 25(24):E5789 (2020); Ferreira, S., et al., Neural Regen Res., June; 16(6):1127-1130 (2021)), places a massive emotional and economic burden on society. Considered one of the fastest growing threat to health, AD is estimated to affect 14 million individuals by 2050 in the United States. The financial encumbrance of AD and other dementias in 2020 was approximately $305 billion and is expected to reach $1.1 trillion by 2050, becoming the most expensive medical disorder globally (FactSheet., Alzheimer's Disease Facts and Figures (2020); Montine, T., et al., Pharmacol Rev., January; 73(1):152-162 (2021)). Despite incremental advancements in the treatment and understanding of its pathophysiology, there is currently no definitive therapy to prevent or slow the course of AD (Zucchella, C. et al., Front. Neurol., 13 December (2018)).

Therefore, there remains a need for improved compositions and methods of use thereof for the treatment of neurodegenerative diseases such as Alzheimer's disease.

It is another object of the invention to provide compositions with higher potency, greater bioavailability, fewer or decreased side effects, or a combination thereof and methods of using them for treating a wide range of neurodegenerative diseases, including Alzheimer's disease.

### SUMMARY OF THE INVENTION

Disclosed herein are cannabidiol based compositions and methods of their use to treat or reduce symptoms associated with neurodegenerative diseases. In one embodiment, the disclosed cannabidiol compositions ameliorate the conditions associated with Alzheimer's disease (AD) and retarded cognitive decline by activation Glial cells and reducing inflammatory indicies. One embodiment provides a method of treating a neurodegenerative disease comprising administering an effective amount of cannabidiol to a subject in need thereof to improved cognitive function and ameliorated the pathophysiology of the neurodegenerative disease. In one embodiment, the pathophysiology of the neurodegenerative disease is associated with β-amyloid peptide (AP) formation and/or aggregation in the subject's brain tissue. In another embodiment, the pathophysiology of the neurodegenerative disease is associated with neuroinflammation in the subject's brain tissue.

In one embodiment, the neurodegenerative disease causes cognitive impairment in the subject having the neurodegenerative disease. In another embodiment, the neurodegenerative disease is Alzheimer's disease.

Also disclosed are findings that CBD increased expression of IL-33 and TREM2, reduced serum IL-6 levels, and ameliorated the symptoms of AD with improved cognitive function in 5xFAD mice. These findings suggest a potential therapeutic role for CBD in the treatment of AD through a bi-directional dialogue with IL-33 and TREM2 signaling. In one embodiment, cannabidiol reduces or inhibits the Aβ formation and/or aggregation in brain tissues of a subject having the neurodegenerative disease. In another embodiment, cannabidiol activates glial cell function to stimulate neuro-protective factors IL-33 and TREM2. In another embodiment, cannabidiol reduces the level of inflammatory cytokines in the subject compared to cytokine levels prior to administration of cannabidiol. In some embodiments cannabidiol reduces the level of inflammatory cytokines including, but not limited to interleukin (IL)-2, IL-7, IL-6, IL-10, tumor necrosis factor (TNF), IFNγ, granulocyte colony-stimulating factor (G-CSF), monocyte chemoattractant protein-1 (MCP1; also known as CCL2), macrophage inflammatory protein 1 alpha (MIP1α; also known as CCL3), CXC-chemokine ligand 10 (CXCL10), C-reactive protein, and ferritin.

Another embodiment provides a pharmaceutical composition comprising an effective amount of a cannabinoid to improve cognitive function and ameliorate the pathophysiology of a neurodegenerative disease in a subject in need thereof. In one embodiment, the composition reduces or inhibits the Aβ formation and/or aggregation in brain tissues of a subject having the neurodegenerative disease. In another embodiment, the composition activates glial cell function to stimulate neuro-protective factors IL-33 and TREM2. In another embodiment, the composition reduces the level of inflammatory cytokines in the subject compared to cytokine levels prior to administration of cannabidiol. In some embodiments cannabidiol reduces the level of inflammatory cytokines including, but not limited to interleukin (IL)-2, IL-7, IL-6, IL-10, tumor necrosis factor (TNF), IFNγ, granulocyte colony-stimulating factor (G-CSF), monocyte chemoattractant protein-1 (MCP1; also known as CCL2), macrophage inflammatory protein 1 alpha (MIP1α; also known as CCL3), CXC-chemokine ligand 10 (CXCL10), C-reactive protein, and ferritin.

In one embodiments, the pharmaceutical composition improves cognitive function and ameliorate the pathophysiology of Alzheimer's disease.

One embodiment provides a pharmaceutical composition formulated for nasal administration. In one embodiment, the pharmaceutical composition is formulated for aerosol administration.

In one embodiment, the cannabinoid is cannabidiol. In another embodiment, the cannabinoid is selected from the group consisting of tetrahydrocannabinols (THC), delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV), cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof. In another embodiment, the cannabinoid is administered in combination with another medicament. In another embodiment, the cannabinoid is administered in combination with a pharmaceutically acceptable excipient.

### BRIEF DESCRIPTION OF THE DRAWINGS

FIGS. 1A-1K show CBD treatment improved cognitive function and ameliorated the pathophysiology of Alzheimer's disease. FIGS. 1A-1C and 1G are immunohistochemical panels (1A-1C) and a bar graph (1G) showing the Novel Object Recognition method (FIGS. 1A-1C). FIGS. 1D-1F and 1H are immunohistochemical panels (1D-1F) and a bar graph (1H) showing the Open Field testing method. FIGS. 1I-1J are immunofluorescence panels of brain tissues of 5xFAD mice treated with CBD. Immunhistochemical panels represent 6-10 animals per each experimental group showing deposition of β-amyloid peptide (Yellow arrows) in the hippocampus area of WT mice brain, and 5xFAD mice brain treated/untreated with CBD. Localization of $-amyloid peptide (RED) on the nucleus is visualized by imposing red staining over blue, creating PINK images. The blue color is representing DNA staining with DAPI (4′,6-diamidino-2-phenylindole) to identify the nuclear presence and cell viability. Images are all shown in 100× magnification.

FIG. 2 shows CBD elevated IL-33 and TREM2 expression in the brain. FIGS. 2A-2L are panels showing immunofluorescence staining of brain tissues from FAD mice treated with CBD and stained with GFAP (green fluorochrome), IL-33 (red fluorochrome). FIGS. 2M-2X are panels showing immunofluorescence staining of brain tissues from FAD mice treated with CBD and stained with IBA1 (green fluorochrome), TREM2 (red fluorochrome). Co-localization of IL-33 or TREM2 (red fluorochrome) with GFAP or IBA1 (green fluorochrome) is visualized by merged panels imposing green over red (yellow) images. The blue color represents DNA staining with DAPI (4′,6-diamidino-2-phenylindole), which depicts nuclei of viable cells. Images are all shown in 100× magnification.

FIGS. 3A-3R show potential protective effects of CBD through enhancement of glial cells function and cytokine modulation. FIGS. 3A-3K flow cytometry panels (3A-3I) and bar graphs (3J-3K) showing the analysis of expression levels of IL-33 (**p≤0.001) and TREM2 (**p≤0.0001) in astrocytes and microglia (GFAP+ and CD11b+ cells) respectively, in brain tissues treated with CBD. FIGS. 3L-3R are flow cytometry panels (3L-3Q) and a bar graph (3R) showing the whole blood analysis of IL-6 expression (**p≤0.009) in CBD-treated mice.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “cannabidiol (CBD)” refers to the major nonpsychotropic cannabinoid compound derived from the plant Cannabis sativa, commonly known as marijuana. CBD has been reported to have antioxidant, anti-inflammatory and neuroprotective effects, which occur independent of the canonical cannabinoid CB1 and CB2 receptors.

### II. Cannabidiol as a Therapeutic Modality for Neurodegenerative Diseases

CBD is non-psychoactive and can trigger apoptosis in immune cells as well as act as an anti-inflammatory agent. This opens new avenues with wide-ranging clinical application in the treatment of inflammatory disease, including neuroinflammatory diseases and disorders.

A. Cannabinoid Compositions

CBD is a safe, non-psychoactive phytocannabinoid produced by cannabis plant. Increasing evidence from the research community suggests an immunomodulatory role for CBD in a variety of inflammatory conditions, potentially including neurodegenerative diseases (Salles É. et al., J Cell Mol Med. November; 24(21):12869-12872 (2020); Hao, F., et al. Life Sci., October 21; 118624 (2020); Mulder, J. et al., Brain. April; 134(Pt 4):1041-60 (2011); Páez J. A. et al., Curr Med Chem. 26(18):3300-3340 (2019)); However, the molecular and cellular mechanisms underlying how CBD may slow neurodegenerative processes are largely unknown.

The term “cannabinoid” as used herein may encompass a chemical compound that activates any mammalian cannabinoid receptor, for example human CB₁ receptor or human CB₂ receptor. The cannabinoids may be naturally occurring (such as, for example, endocannabinoids or phytocannabinoids) or they may be synthetic. Synthetic cannabinoids may include, for example, the classical cannabinoids structurally related to THC, the non-classical cannabinoids (cannabimimetics) including the aminoalkyindoles, 1,5-diarylpyrazoles, quinolines and arylsulphonoamides, and eicosanoids related to the endocannabinoids. When a cannabinoid salt is used, it may be employed in the form of a solution. The one or more cannabinoids is preferably selected from the classical cannabinoids, more preferably selected from tetrahydrocannabinols (THC), preferably delta-9-tetrahydrocannabinol and delta-8-tetrahydrocannabinol, cannabidiol (CBD), cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV and cannabigerol monomethyl ether (CBGM). CBD is a preferred cannabinoid.

Other cannabinoids suitable for use in the present invention are endocannabinoids, substances that naturally occur in the mammalian body and which activate one or more cannabinoid receptor. Preferably endocannabinoids are selected from arachidonoylethanolamine (AEA), 2-arachidonoylglycerol (2-AG), 2-arachidonyl glyceryl ether (noladin ether), N-arachidonoyl dopamine (NADA), virodhamine (OAE) and lysophosphatidylinositol (LPI).

Synthetic cannabinoids suitable for use in the present invention include nabilone, rimonabant, JWH-073, CP-55940, dimethylheptylpyran, HU-210, HU-331, SR144528, WIN 55,212-2, JWH-133, levonantradol, and AM-2201.

In recent years, cannabinoids have been investigated extensively due to their potential effects on the human body. Among all cannabinoids, Cannabidiol (CBD) has demonstrated a potent anti-inflammatory effect in a variety of inflammatory conditions. In one embodiment, CBD is used to activate neuroprotective factors and improved cognitive function and ameliorated the pathophysiology of Alzheimer's disease. In another embodiment, the administration of CBD regulated IL-33 and TREM2 expression to activate neuroprotection from neurodegenerative diseases. In another embodiment, the neurodegenerative disease is Alzheimer's disease.

In another embodiment, the administration of CBD downregulates the level of pro-inflammatory cytokines and ameliorates the clinical symptoms of neuroinflammatory diseases.

B. Intranasal Compositions

In one embodiment, the cannabidiol compositions are formulated to allow intranasal administration. Intranasal compositions may comprise an inhalable dry powder pharmaceutical formulation comprising a therapeutic agent, wherein the therapeutic agent is present as a freebase or as a mixture of a salt and a freebase. Pharmaceutical formulations disclosed herein can be formulated as suitable for airway administration, for example, nasal, intranasal, sinusoidal, peroral, and/or pulmonary administration. Typically, formulations are produced such that they have an appropriate particle size for the route, or target, of airway administration. As such, the formulations disclosed herein can be produced so as to be of defined particle size distribution.

For example, the particle size distribution for a salt form of a therapeutic agent for intranasal administration can be between about 5 μm and about 350 μm. More particularly, the salt form of the therapeutic agent can have a particle size distribution for intranasal administration between about 5μ to about 250 μm, about 10 μm to about 200 μm, about 15 μm to about 150 μm, about 20 μm to about 100 μm, about 38 μm to about 100 μm, about 53 μm to about 100, about 53 μm to about 150 μm, or about 20 μm to about 53 μm. The salt form of the therapeutic agent in the pharmaceutical compositions of the invention can a particle size distribution range for intranasal administration that is less than about 200 μm. In other embodiments, the salt form of the therapeutic agent in the pharmaceutical compositions has a particle size distribution that is less than about 150 μm, less than about 100 μm, less than about 53 μm, less than about 38 μm, less than about 20 μm, less than about 10 μm, or less than about 5 μm. The salt form of the therapeutic agent in the pharmaceutical compositions of the invention can have a particle size distribution range for intranasal administration that is greater than about 5 μm, greater than about 10 μm, greater than about 15 μm, greater than about 20 μm, greater than about 38 μm, less than about 53 μm, less than about 70 μm, greater than about 100 μm, or greater than about 150 μm.

Additionally, the salt form of the therapeutic agent in the pharmaceutical compositions of the invention can have a particle size distribution range for pulmonary administration between about 1 μm and about 10 μm. In other embodiments for pulmonary administration, particle size distribution range is between about 1 μm and about 5 μm, or about 2 μm and about 5 μm. In other embodiments, the salt form of the therapeutic agent has a mean particle size of at least 1 μm, at least 2 μm, at least 3 μm, at least 4 μm, at least 5 μm, at least 10 μm, at least 20 μm, at least 25 μm, at least 30 μm, at least 40 μm, at least 50 μm, at least 60 μm, at least 70 μm, at least 80 μm, at least 90 μm, or at least 100 μm.

In some embodiments the disclosed cannabinoid compositions include one or more cannabinoids or pharmaceutically acceptable derivatives or salts thereof, a propellant, an alcohol, and a glycol and/or glycol ether. The alcohol may be a monohydric alcohol or a polyhydric alcohol, and is preferably a monohydric alcohol. Monohydric alcohol has a lower viscosity than a glycol or glycol ether. Accordingly, the composition is able to form droplets of a smaller diameter in comparison to compositions in which the monohydric alcohol is not present. The present inventors have surprisingly found that a specific ratio of monohydric alcohol to glycol or glycol ether results in a composition with a desired combination of both long term stability (for example the composition remains as a single phase for at least a week at a temperature of 2-40° C.) and small droplet size.

### III. Methods for Treating Neurodegenerative Diseases

The present invention relates to the treatment of neurodegenerative diseases, disorders, and/or conditions, with cannabidiol compositions. Methods and compositions for application are provided. In particular, the invention activates neuroprotective factors in an individual diagnosed with, or at risk of having, or suspected of having a neurodegenerative disease, disorder, and/or condition. Methods of administering a therapeutic agent are provided. In particular, the present invention provides methods of administering cannabidiol to the central nervous system (CNS) for the activation of neuroprotective factors and suppressing pro-inflammatory factors to prevent, inhibit or treat neurodegenerative diseases.

Neurodegenerative diseases to be treated can include any disease or disorder or symptoms or causes or effects thereof involving the damage or deterioration of neurons. Neurodegenerative diseases can include, but are not limited to, Alexander Disease, Alper's Disease, Alzheimer Disease, Amyotrophic Lateral Sclerosis, Ataxia Telangiectasia, Canavan Disease, Cockayne Syndrome, Corticobasal Degeneration, Creutzfeldt-Jakob Disease, Huntington Disease, Kennedy's Disease, Krabbe Disease, Lewy Body Dementia, Machado-Joseph Disease, Multiple Sclerosis, Parkinson Disease, Pelizaeus-Merzbacher Disease, Niemann-Pick's Disease, Primary Lateral Sclerosis, Refsum's Disease, Sandhoff Disease, Schilder's Disease, Steele-Richardson-Olszewski Disease, Tabes Dorsalis or any other condition associated with damaged neurons. Other neurodegenerative conditions can include or be caused by traumatic spinal cord injury, ischemic spinal cord injury, stroke, traumatic brain injury, and hereditary conditions.

Brain diseases such as neurodegenerative diseases and neuroinflammatory disorders are devastating conditions that affect a large subset of the population. Many are presently incurable, highly debilitating, and often result in progressive deterioration of brain structure and function over time. Disease prevalence is also increasing rapidly due to growing aging populations worldwide, since the elderly are at high risk for developing these conditions. Currently, many neurodegenerative diseases and neuroinflammatory disorders are difficult to diagnose due to limited understanding of the pathophysiology of these diseases. Meanwhile, current treatments are ineffective and do not meet market demand; demand that is significantly increasing each year due to aging populations.

For this reason, a method is disclosed that involves administration of cannabidiol to a subject in need thereof to activate of neuroprotective factors and suppress pro-inflammatory factors for the treatment of neurodegenerative diseases. This can involve administering an effective amount of a pharmaceutical composition having cannabidiol to the CNS of a mammal in need of. The method provides neuroprotective and neurorestorative effects of a cannabidiol compositions for patients with neurodegenerative diseases, such as Alzheimer's disease.

A. Alzheimer's Disease

Alzheimer's disease is a complex neurodegenerative disorder that is a associated with irreversible cognitive decline in late middle or old age. In 2016, about 47 million people were reported to suffer from dementia, a tremendous number that is predicted to increase to more than 131 million by 2050 (Prince et al. Alzheimer's Disease International (2016)). AD is the only disease among the top 10 potentially fatal diseases for which no treatments are available. Common symptoms of AD include dementia, progressive memory loss, dysfunctional thoughts, confusion, and changes in both traits and moods. It has long been thought that neuronal accumulation of Aβ, a phenotype found in brain tissues of AD patients, is the main cause of AD (Bahmanyar et al. Science. 237(4810):77-80 (1987)). Although the primacy of Aβ in the etiology of AD has recently come into question, there is still a general consensus that Aβ is closely related to AD. Typically, this usually means that AD is studied in the context of Aβ and neurons. However, recent research has also considered AD in the context of glial cells, reflecting the growing interest in the pathophysiological role of these cells in the brain (Cairns et al. Neurosci Lett. 230:49-52 (1997)). Most such studies have focused on microglia, the primary immune glial cell type in the brain. However, astrocytes—another glial cell type—are also involved in the immune system of the brain and in maintaining brain homeostasis. Accordingly, extending studies of AD to include a consideration of both microglia and astrocytes opens new avenues for identifying novel targets for AD treatment.

B. Neuroinflammation

Inflammation is a defense reaction against diverse insults, designed to remove noxious agents and to inhibit their detrimental effects. It consists of molecular and cellular mechanisms and an intricate network of controls to keep them in check. In neurodegenerative diseases, neuroinflammation may be triggered by the accumulation of aggregated or otherwise modified proteins, by signals emanating from injured neurons, or by imbalances between pro- and anti-inflammatory processes.

In addition to protein aggregation, neuroinflammatory changes are present in AD brains, including alterations in the morphology, activation and distribution of microglia and astrocytes (microgliosis and astrogliosis) as well as increased expression of inflammatory mediators (Beach, T. G. et al., Glia. 2:420-36 (1989); Itagaki, S. et al., J Neuroimmunol. 24:173-82 (1989); McGeer, P. L. et al., Neurosci Lett. 79:195-200 (1987)). However, the exact contributions of both microgliosis and astrogliosis in AD are not clear. While first hypothesized to contribute to AD neuropathology, gliosis and neuroinflammation seem to have more complex effects and could be either beneficial or damaging in those with AD (Wyss-Coray T. et al., Neuron. 35:419-32 (2002)). For example, reactive microglia and astrocytes can contribute to the clearance of Aβ. Conversely, the production of pro-inflammatory cytokines like TNFα (Tumor necrosis factor α) or IL1-β (Interleukin 1β) resulting from glial activation are harmful and toxic to neurons. Many studies also suggest that neuroinflammation exacerbates tau phosphorylation. Altogether, these data suggest the possibility that gliosis and neuroinflammation have neuroprotective roles early in AD by controlling amyloid load, but later can become toxic to neurons and act as a catalyst for neurodegeneration (Gratuze et al, Mol. Mol Neurodegeneration 13, 66 (2018)).

1. Glial Cells

Glial cells are by far the most numerous cells in the brain, outnumbering neurons by about 10 to 1. These cells, which come in various forms—such as microglia, astrocytes, and oligodendrocytes—surround and support the function and healthy of neurons. For example, microglia protect neurons from physical and chemical damage and are responsible for clearing foreign substances and cellular debris from the brain. To carry out these functions, glial cells often collaborate with blood vessels in the brain. Together, glial and blood vessel cells regulate the delicate balance within the brain to ensure that it functions at its best.

Research suggests that chronic inflammation may be caused by the buildup of glial cells normally meant to help keep the brain free of debris. One type of glial cell, microglia, engulfs and destroys waste and toxins in a healthy brain. In Alzheimer's, microglia fail to clear away waste, debris, and protein collections, including Aβ plaques.

2. TREM-2

TREM2 belongs to a family of receptors referred to as the triggering receptors expressed on myeloid cells (TREM). Members of the TREM family are cell surface transmembrane glycoproteins with V-immunoglobulin extracellular domains and cytoplasmic tails. The TREM2 gene is located on human chromosome 6p21 and encodes a 230-amino acid glycoprotein. The TREM2 gene is expressed in a subgroup of myeloid cells including dendritic cells, granulocytes, and tissue-specific macrophages like osteoclasts, Kuppfer cells and alveolar macrophages. In the brain, TREM2 is exclusively expressed by microglia; however, there is some discordance regarding the level of its expression/translation and whether or not TREM2 is present in all or only a subgroup of microglia in mice and humans. Interestingly, the expression of TREM2 varies depending on the particular region of the central nervous system (CNS), with a higher expression in the hippocampus, the spinal cord and the white matter. TREM2 expression is up regulated in pathological conditions such as Parkinson's disease (PD), Amyotrophic lateral sclerosis (ALS), stroke, traumatic brain injury and AD. In AD, increased expression of TREM2 has been confirmed in patients and in mouse models of amyloid and tau pathology and seems to be associated with the recruitment of microglia to amyloid plaques. Research indicates that the acute inflammation mimicked by in vitro studies first induces a decrease of TREM2 expression while chronic inflammation observed in pathological conditions, such as AD, results in an increase of TREM2 expression (Gratuze et al, Mol. Mol Neurodegeneration 13, 66 (2018)).

TREM2's association with Alzheimer's disease supports the involvement of immune and inflammatory pathways in the cause of the disease, rather than as a consequence of the disease. TREM2 variants associated with Alzheimer's disease induce partial loss of function of the TREM2 protein and alter the behavior of microglial cells, including their response to amyloid plaques. TREM2 variants have also been shown to cause polycystic lipomembranous osteodysplasia with sclerosing leukoencephalopathy and frontotemporal dementia.

Normally, TREM2 tells the microglia cells to clear β-amyloid peptide plaques from the brain and helps fight inflammation in the brain. In the brains of people where this gene does not function normally, plaques build up between neurons. Astrocytes, another type of glial cell, are signaled to help clear the buildup of plaques and other cellular debris left behind. These microglia and astrocytes collect around the neurons but fail to perform their debris-clearing function. In addition, they release chemicals that cause chronic inflammation and further damage the neurons they are meant to protect.

3. IL-33

Chronic neuroinflammation induced by microglia contributes to pathological progression and symptom severity in late stages of the disease. Importantly, although sometimes contradictory, modulation of the immune system by specific interleukin signals such as IL-12/IL-23 and IL-10, as well as the inflammasome pathway, ameliorates AD-like pathology (Vom Berg J, et al., Nat Med 18(12):1812-1819 (2012)).

IL-33, an alarmin of the IL-1 family, is a crucial mediator of the innate immune response and a regulator of immune cell infiltration and activation. IL-33 is expressed in various cell types (Liew F Y, Nat Rev Immunol 10(2):103-110 (2010)). IL-33 binds to a heterodimeric receptor complex, comprising ST2 and IL-1RAcP, and triggers the intracellular cascade involve myeloid differentiation factor 88 (MyD88) and NF-κB. This signaling selectively activates type 2 T-helper cells, mast cells, neutrophils, and alternatively-activated macrophages. IL-33 exhibits the highest expression levels within the central nervous system (CNS) (Liew F. Y. et al., Nat Rev Immunol. November; 16(11):676-689 (2016)). IL-33 is constitutively produced by glial cells and following damaged to the blood-brain barrier cells (Liew F. Y. et al., Nat Rev Immunol. November; 16(11):676-689 (2016)). Importantly, IL-33 expression is reduced in AD patients (Saresella, M. et al., J Neuroinflammation., June 6; 17(1):174 (2020); Liang C. S. et al., Alzheimers Res Ther. July 16; 12(1):86 (2020); Abd Rachman Isnadi M. F. et al., Mediators Inflamm. February 4; 2018:5346413 (2018)), suggesting a possible role as a biomarker and/or a novel therapeutic target for AD.

IL-33 is a pleiotropic cytokine with diverse functions in various infectious and inflammatory diseases that may exert beneficial or detrimental effects on the disease ((Liew F Y, Nat Rev Immunol 10(2):103-110 (2010)). First, it has an important protective function against infection. Mice infected with the influenza virus exhibit reduced pulmonary inflammation and pathology after IL-33 treatment ((Liew F Y, Nat Rev Immunol 10(2):103-110 (2010)). IL-33 mRNA levels are elevated in the colon of Trichuris muris-resistant mice (Humphreys et al. Journal of immunology 180, 2443-2449 (2008)). In contrast, IL-33 promotes the pathogenesis of T helper 2 (Th2) cell-related diseases including asthma, atopic dermatitis, and anaphylaxis by inducing cytokine production in Th2 cells and other innate immune cells (Kakkar, R. & Lee, R. T. Nature reviews Drug discovery 7, 827-840 (2008)). For example, asthma patients have higher IL-33 expression levels; concordantly, IL-33 administration exacerbates asthma pathology in mouse models of asthma (Fu, A. K. Y. et al., PNAS 113 (19) E2705-E2713 (2016)).

Genetic studies have identified three single nucleotide polymorphisms in the IL-33 gene that are associated with a reduced risk of AD development in Caucasian populations (Chapuis et al. Mol Psychiatry 14, 1004-1016 (2009)). One of the SNPs, rsl 179633, is strongly associated with late-onset of Alzheimer's disease in Chinese populations (Yu et al. Neurobiology of aging 33, 1014 elOl 1-1014 (2012)). Additional evidence of the involvement of IL-33 in AD comes from the finding that IL-33 mRNA expression is decreased in human AD brains (Chapuis et al. Mol Psychiatry 14, 1004-1016 (2009)). There is also evidence that IL-33 and ST2-positive cells are significantly increased in the entorhinal cortex of AD patients, although this remains controversial.

Furthermore, IL-33 and ST2 co-localize with amyloid plaques and neurofibrillary tangles in the brains of AD patients. In the aggregates, despite a discrepancy between mRNA and protein expression in AD brains, these findings suggest that the regulation of IL-33/ST2 signaling is important in AD pathology.

4. IL-6

Interleukin-6 (IL-6) is a pleiotropic inflammatory cytokine mainly produced by activated microglia, astrocytes in different brain regions. In addition, IL-6 could stimulate microglia and astrocytes to release a cascade of proinflammatory cytokines and acute-phase proteins, such as C-reactive protein (CRP). The levels of IL-6 have been found significantly elevating in the brains, cerebrospinal fluid, and plasma, especially locally around amyloid plaques in AD patients and animal models (Wang, W-Y., et al., Ann Transl Med. June; 3(10): 136 (2015)). Therefore, it has been proposed IL-6 is involved in the etiopathology of AD with acute or chronic inflammatory components.

A number of studies have investigated the molecular mechanism underlying the association of IL-6 with AD including tau and Aβ. Production of IL-6 by human neurons is reportedly stimulated by glycation end product-modified tau and Aβ.

IL-6 in neuroinflammation and neurodegeneration also plays a complex role in regulating cognitive function. As early as 2002, Weaver et al. has found that elevated IL-6 correlated with age-related cognitive decline in humans (Weaver, J. D. et al. Neurology 59:371-8 (2002)). Subsequently, many researchers confirmed that under inflammatory conditions, excessive IL-6 through activation of neuronal NADPH-oxidase induced by aging or inflammation may impair cognitive processes, such as spatial learning and memory.

### IV. Administration

Any route of CBD administration may be employed so long as that route and the amount administered are therapeutically useful. Inhalation is a convenient administration route for therapeutic agents that overcomes many of the drawbacks of oral administration, such as slow drug onset and first-pass metabolism plus it can be used with patients that suffer from pulmonary conditions.

A. Intranasal Administration

In one embodiment, the CBD compositions are delivered through intranasal administration. As described herein, intranasal administration or nose administration comprise the described compositions being administered into the mammal nostril and reaching nasal meatus or nasal cavity. For example, the compositions can be administered with nasal spray, insufflation, nasal drop, aerosol, propellant, pressurized dispersion body, aqueous aerosol, propellant, nose suspension, instillation, nasal gel, nose is with ointment and nose ointment, by means of any new or old type equipment of administration.

B. Intrathecal and Intracranial Administration

In one example, routes of administration to the CNS include intrathecal and intracranial. Intracranial administration may be to the cisterna magna or ventricle. The term “cisterna magna” is intended to include access to the space around and below the cerebellum via the opening between the skull and the top of the spine. The term “cerebral ventricle” is intended to include the cavities in the brain that are continuous with the central canal of the spinal cord. The CBD delivered in the intrathecal methods of treatment of the present invention may be administered through any convenient route commonly used for intrathecal administration. For example, the intrathecal administration may be via a slow infusion of the formulation for about an hour.

The intrathecal administration of the present invention may comprise introducing the composition into the lumbar area. Any such administration may be via a bolus injection. Depending on the severity of the symptoms and the responsiveness of the subject to the therapy, the bolus injection may be administered once per week, once per month, once every 6 months or annually.

In other embodiments, the intrathecal administration is achieved by use of an infusion pump. Those of skill in the art are aware of devices that may be used to effect intrathecal administration of a composition. The composition may be intrathecally given, for example, by a single injection, or continuous infusion. It should be understood that the dosage treatment may be in the form of a single dose administration or multiple doses.

As used herein, the term “intrathecal administration” is intended to include delivering a pharmaceutical composition directly into the cerebrospinal fluid of a subject, by techniques including lateral cerebroventricular injection through a burrhole or cisternal or lumbar puncture or the like. The term “lumbar region” is intended to include the area between the third and fourth lumbar (lower back) vertebrae and, more inclusively, the 2-S1 region of the spine.

C. Injection Administration

Administration of a composition in accordance with the present invention to any of the above mentioned sites can be achieved by direct injection of the composition or by the use of infusion pumps. For injection, the composition can be formulated in liquid solutions, e.g., in physiologically compatible buffers such as Hank's solution, Ringer's solution or phosphate buffer. In addition, the enzyme may be formulated in solid form and re-dissolved or suspended immediately prior to use. Lyophilized forms are also included. The injection can be, for example, in the form of a bolus injection or continuous infusion (e.g., using infusion pumps) of the enzyme.

In one embodiment of the invention, CBD is administered by intracerebroventricular (icv) injection into the brain of a subject. The injection can be made, for example, through a burr hole made in the subject's skull. In another embodiment, the enzyme and/or other pharmaceutical formulation is administered through a surgically inserted shunt into the cerebral ventricle of a subject. For example, the injection can be made into the lateral ventricles, which are larger, even though injection into the third and fourth smaller ventricles can also be made. In yet another embodiment, the compositions used in the present invention are administered by injection into the cisterna magna or lumbar area of a subject.

The composition containing CBD may be in the form of an injectable unit dose. Examples of carriers or diluents usable for preparing such injectable doses include diluents such as water, ethyl alcohol, macrogol, propylene glycol, ethoxylated sostearyl alcohol, polyoxyisostearyl alcohol and polyoxyethylene sorbitan fatty acid esters, pH adjusting agents or buffers such as sodium citrate, sodium acetate and sodium phosphate, stabilizers such as sodium pyrosultite, EDTA, thioglycolic acid and thiolactic acid, isotonic agents such as sodium chloride and glucose, local anesthetics such as procaine hydrochloride and lidocaine hydrochloride. Furthermore, usual solubilizing agents and analgesics may be added. Injections can be prepared by adding such carriers to the enzyme or other active, following procedures well known to those of skill in the art. A thorough discussion of pharmaceutically acceptable excipients is available in REMINGTON'S PHARMACEUTICAL SCIENCES (Mack Pub. Co., N.J. 1991). The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

D. Formulations

Compositions described herein may be employed in combination with another medicament. The compositions can appear in conventional forms, for example, aerosols, solutions, suspensions, or topical applications, or in lyophilized form.

Typical compositions include CBD and a pharmaceutically acceptable excipient which can be a carrier or a diluent. For example, the active agent(s) may be mixed with a carrier, or diluted by a carrier, or enclosed within a carrier. When the active agent is mixed with a carrier, or when the carrier serves as a diluent, it can be solid, semi-solid, or liquid material that acts as a vehicle, excipient, or medium for the active agent. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active agent(s). Such additives can include wetting agents, emulsifying and suspending agents, salt for influencing osmotic pressure, buffers and/or coloring substances preserving agents, sweetening agents or flavoring agents. The compositions can also be sterilized if desired.

If a liquid carrier is used, the preparation can be in the form of a liquid such as an aqueous liquid suspension or solution. Acceptable solvents or vehicles include sterilized water, Ringer's solution, or an isotonic aqueous saline solution.

The agent(s) may be prodded as a powder suitable for reconstitution with an appropriate solution as described above. Examples of these include, but are not limited to, freeze dried, rotary dried or spray dried powders, amorphous powders, granules, precipitates, or particulates. The composition can optionally contain stabilizers, pH modifiers, surfactants, bioavailability modifiers and combinations of these. A unit dosage form can be in individual containers or in multi-dose containers.

In one embodiment, the preparation can contain an agent, dissolved or suspended in a liquid carrier, such as an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens. For example, in addition to solubility, efficient delivery to the CNS following intranasal administration may be dependent on membrane permeability. For enzymes where paracellular transport is hindered due to size and polarity, improving membrane permeability may enhance extracellular mechanisms of transport to the CNS along olfactory and trigeminal nerves. One approach to modifying membrane permeability within the nasal epithelium is by using permeation enhancers, such as surfactants, e.g., lauroylcarnitine (LC), bile salts, lipids, cyclodextrins, polymers, or tight junction modifiers.

Generally, the active agents are dispensed in unit dosage form including the active ingredient together with a pharmaceutically acceptable carrier per unit dosage. Usually, dosage forms suitable for nasal administration include from about 125 μg to about 125 mg, e.g., from about 250 μg to about 50 mg, or from about 2.5 mg to about 25 mg, of the compounds admixed with a pharmaceutically acceptable carrier or diluent.

The CBD delivered in the intranasal methods of treatment of the present invention may be administered in suitable dose ranges, generally about 0.1 to 10 mg/kg/day.

Dosage forms can be administered daily, or more than once a day, such as twice or thrice daily. Alternatively, dosage forms can be administered less frequently than daily, such as every other day, or weekly, if found to be advisable by a prescribing physician.

### EXAMPLES

### Example 1: CBD Treatment Improved Cognitive Function and Ameliorated the Pathophysiology of Alzheimer's Disease

Methods and Materials

Animals and Experimental Groups.

5xFAD (expressing human APP and PSEN1 transgenes with a total of five AD-linked mutations) transgenic mice (male 9-12-month-olds) were used. These 5xFAD transgenic mice rapidly recapitulate major features of AD amyloid pathology. Mice were randomized into two experimental groups (placebo or CBD-treated; 6-10 mice/group) by a blinded investigator. A group of untreated wild-type (WT) experiments were performed in accordance with the rules and regulations of the Augusta University Institutional Animal Care and Use Committee (IACUC). In the treatment group, mice received CBD isolate (tetrahydrocannabinol-free, Canabidiol Ltd., Dublin, Ireland), 10 mg/kg intraperitoneally every other day for 8 doses over 2 weeks. Placebo-treated mice were administered along the same schedule as CBD.

Behavioral Tests

To assess the behavior, Open Field (OF) and Novel Object Recognition (NOR) tests were used. For NOR, mice were placed in an enclosed box with two identical objects that were placed within a 10 cm circle, at a set distance apart. Mice were then removed from the environment for a predetermined amount of time, and one of the two previously used (familiar) objects was replaced with a novel object that was different from the familiar object in shape, texture, and appearance. The ability of the mouse to discriminate between the familiar and novel object was quantified as a discrimination index, DI=(Tn−Tf)/(Tn+Tf), where Tn is the time spent by the mouse with the novel object, and Tf indicates the time spent with the familiar object. In OF evaluation, mice were tested in a square box (40 cm by 40 cm by 40 cm) for 10 min, and activity was digitally recorded. Distance traveled, mean velocity, and time spent in the center zone were analyzed with Ethovision XT video-tracking software (Noldus Information Technology, USA).

Histology and Immunofluorescence Staining

Brain and blood tissues were collected and processed for histology and Immunofluorescence imaging. Briefly, brain tissues were labeled with specific, fluorescent-conjugated antibodies with fluorescent conjugation against amyloid-β, IL-33, IBA1 (for microglia), TREM2, and GFAP (Glial Fibrillary Acidic Protein for astrocytes identification). Slides were counterstained using DAPI (4′,6-diamidino-2-phenylindole) prior to examination and imaging by Zeiss Fluorescence Microscope.

Results

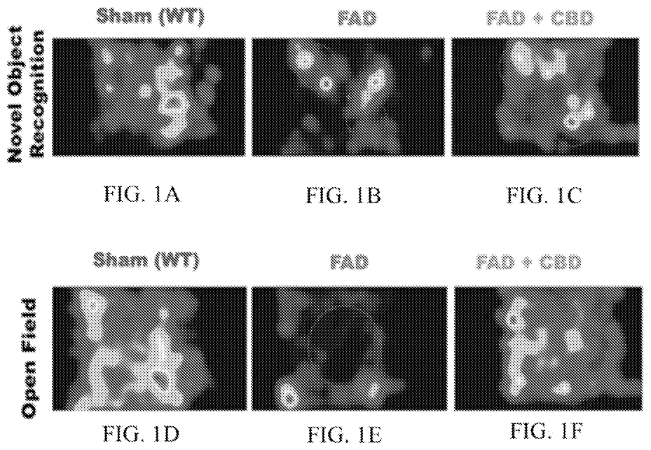

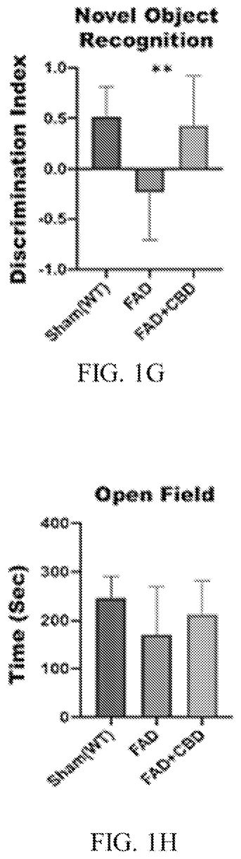

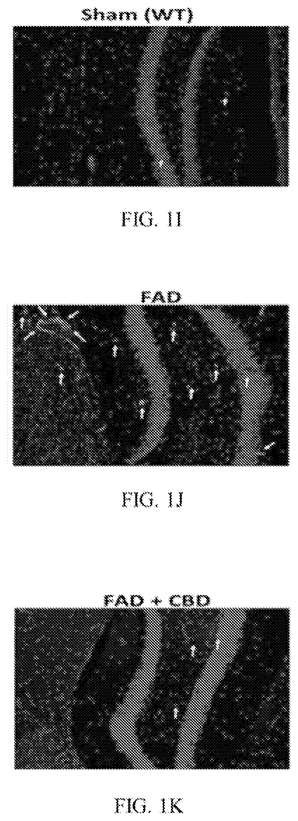

CBD treatment improved cognitive function as measured by NOR (DI increased to 0.5±0.9 from −0.2±0.8, p≤0.04) and OF test (Central zone stay increased to 250±80 sec from 190±90 sec, p>0.05) (FIGS. 1A-1H). The Open Field testing method (FIGS. 1D-1F and 1H) showed that CBD treatment could ameliorate the cognitive function in 5xFAD mice. Further, immunofluorescence staining demonstrated the reduction of β-amyloid peptide in brain tissues of CBD treated 5xFAD mice (FIGS. 1I-1K), indicating the protective effects and potential reduction in the pathophysiology of AD.

### Example 2: CBD Elevated IL-33 and TREM2 Expression in the Brain

Methods and Materials

Analytical Flow Cytometry

For flow cytometry analysis, single-cell suspension was prepared from brain, and blood by sieving the brain tissues through a 100 M cell strainer followed by centrifugation (252 g, 10 min). All cells were then stained with fluorescent antibodies based on routine flow cytometry staining protocol as described previously [6] to quantify astrocytes (GFAP+cells), IL-33, myeloid cells (CD11b+), and TREM2 in the brain as well as leukocytes (CD45+) and IL-6 in the blood. IL-6 was measured as a reliable biomarker in the blood. Cells were then washed and run through a 4-Laser LSR II flow cytometer. Flow cytometry data were analyzed using FlowJo V10. Proper compensation was set to ensure the median fluorescence intensities of negative and positive cells were identical and then was used to gate the population. Gating excluded dead cells and debris using forward and side scatter plots. To confirm the specificity of primary antibody binding and rule out nonspecific Fc receptor binding to cells or other cellular protein interactions, negative control experiments were conducted using isotype controls matched to each primary antibody's host species, isotype, and conjugation format.

Statistical Analysis

Brown-Forsythe and Welch ANOVA were used to establish significance (p<0.05) among groups and for statistical analysis.

Results

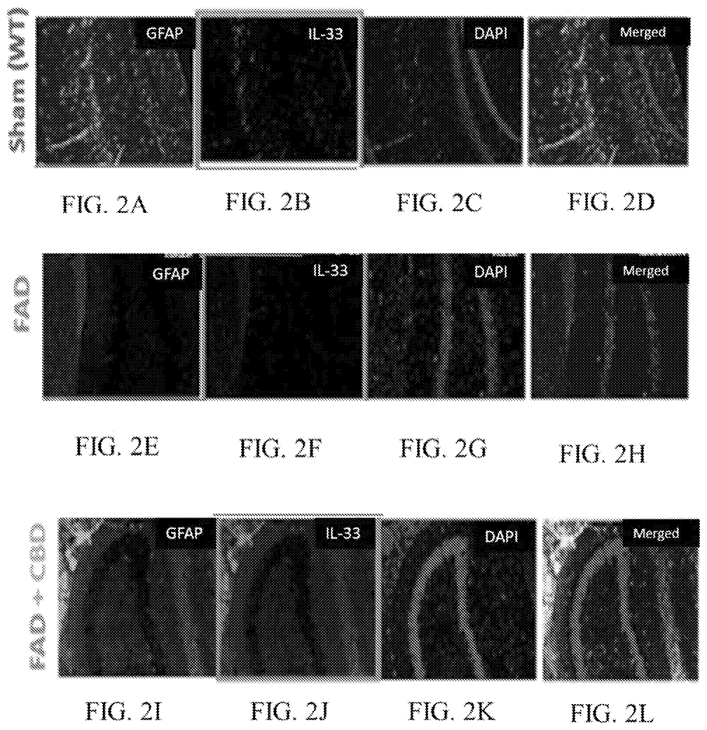

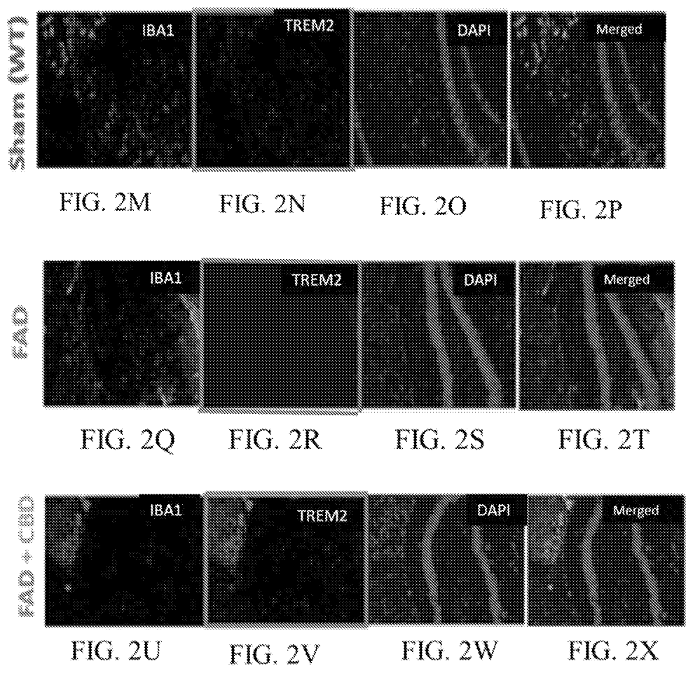

Additional Immunofluorescence staining displayed elevation of IL-33 and TREM2 in the brain of CBD treated mice compared to untreated group (FIGS. 2A-2X). FIGS. 2A-2L showed that CBD treatment was able to increase the expression of IL-33 (FIG. 2J) in the brain tissues of 5xFAD mice, which is specifically more evident in astrocytes (FIG. 2I). CBD treatment demonstrated an increase in TREM2 expression (FIG. 2V) in the brain tissues of 5xFAD mice, distinctively in microglial cells (FIG. 2U). Co-localization of IL-33 or TREM2 with GFAP or IBA1 is visualized by merged panels.

### Example 3: Protective Effects of CBD Through Enhancement of Glial Cells Function and Cytokine Modulation

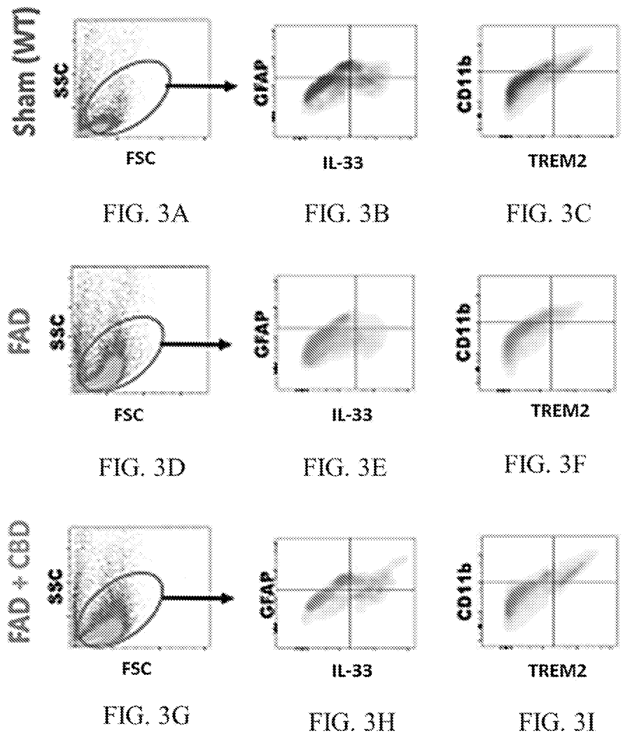

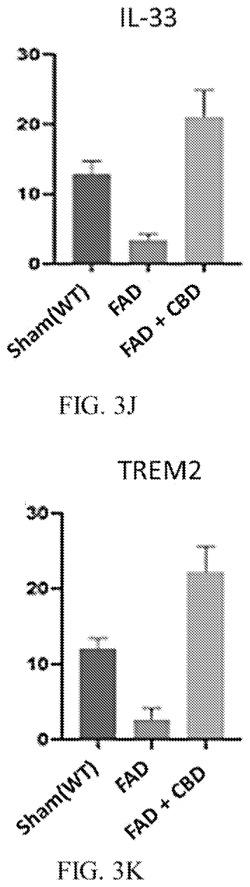

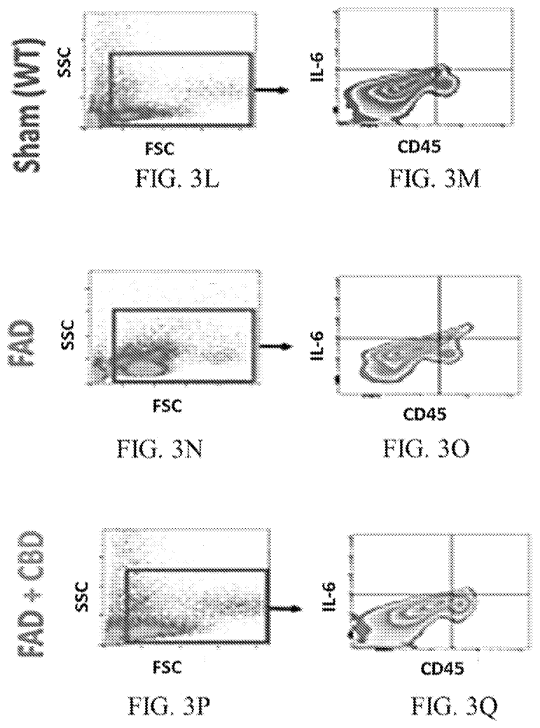

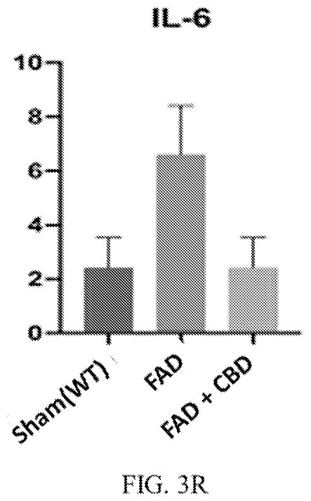

Flow cytometry showed 7-fold increase in IL-33 (FIGS. 3H, 3B; p≤0.001), 10-fold increase in TREM2 (FIGS. 3I, 3C; p≤0.0001) expression in astrocytes and microglia (GFAP+ and CD11b+ cells) respectively, while decreasing IL-6 expression in peripheral leukocytes from 6.9±1.9% to 2.1±1.4% (p≤0.009) (FIGS. 3Q, 3R). This data shows that CBD has a potent immunomodulatory function and shows IL-6 as a reliable diagnostic and prognostic biomarker in the course of AD.

All currently approved treatments for AD mitigate symptoms rather than directly address the underlying pathophysiological processes. The findings presented herein identify a direct therapeutic benefit of CBD in an experimental model of early-onset familial AD. Consistent with putative protective roles for IL-33 and TREM2 in AD (Saresella, M. et al., J Neuroinflammation., June 6; 17(1):174 (2020); Gratuze et al, Mol. Mol Neurodegeneration 13, 66 (2018)), the data presented herein shows that CBD dramatically elevated IL-33 and TREM2 expression in glial cells while suppressing pro-inflammatory IL-6 expression in peripheral blood leukocytes in proportion to cognitive improvements. Interestingly, TREM2 is implicated in microglial phagocytosis; thus, it is notable that CBD increased TREM2 expression in parallel to reduced β-amyloid peptide within brain tissue, suggesting CBD may reduce β-amyloid peptide production and/or aid in clearance of β-amyloid peptide. Similarly, CBD stimulated astrocytic expression of IL-33, a cytokine that enhanced microglial mediated phagocytosis of β-amyloid peptide and improved contextual memory in a mouse mode of AD. Thus, CBD is a clinically-safe and efficacious disease-modifying therapy that attenuates neurocognitive decline, at least in part, via modulation of glial cell function. All currently approved treatments for AD mitigate symptoms rather than directly address the underlying pathophysiological processes. The findings presented herein identify a direct therapeutic benefit of CBD in an experimental model of early-onset familial AD. Consistent with putative protective roles for IL-33 and TREM2 in AD (Saresella, M. et al., J Neuroinflammation., June 6; 17(1):174 (2020); Gratuze et al, Mol. Mol Neurodegeneration 13, 66 (2018)), CBD dramatically elevated IL-33 and TREM2 expression in glial cells while suppressing pro-inflammatory IL-6 expression in peripheral blood leukocytes in proportion to cognitive improvements. Interestingly, TREM2 is implicated in microglial phagocytosis; thus, it is notable that CBD increased TREM2 expression in parallel to reduced β-amyloid peptide within brain tissue, suggesting CBD may reduce β-amyloid peptide production and/or aid in clearance of β-amyloid peptide. Similarly, CBD stimulated astrocytic expression of IL-33, a cytokine that enhanced microglial mediated phagocytosis of β-amyloid peptide and improved contextual memory in a mouse mode of AD. Thus, CBD represents a clinically-safe and efficacious disease-modifying therapy to attenuate neurocognitive decline, at least in part, via modulation of glial cell function.

We claim:

1. A method of treating a neurodegenerative disease comprising administering an effective amount of cannabidiol to a subject in need thereof to improve cognitive function and ameliorate the pathophysiology of the neurodegenerative disease.

2. The method of claim 1, wherein β-amyloid formation and aggregation in the subject's brain tissue is associated with pathophysiology of the neurodegenerative disease.

3. The method of claim 1, wherein a neuroinflammation in the subject's brain tissue is associated with pathophysiology of the neurodegenerative disease.

4. The method of claim 1, wherein the neurodegenerative disease causes cognitive impairment in the subject.

5. The method of claim 1, wherein the neurodegenerative disease is Alzheimer's disease.

6. The method of claim 1, wherein the cannabidiol reduces or inhibits the β-amyloid formation and aggregation in brain tissues of a subject having the neurodegenerative disease.

7. The method of claim 1, wherein the cannabidiol activates glial cell function to stimulate neuroprotective factors.

8. The method of claim 1, wherein the neuroprotective factors are IL-33 and TREM2.

9. The method of claim 1, wherein the cannabidiol in the subject reduces the level of inflammatory cytokines compared to cytokine levels prior to administration of cannabidiol.

10. The method of claim 9, wherein the inflammatory cytokines are selected from the group consisting of IL-6, IFNγ and TNFα and combinations thereof.

11. A pharmaceutical composition comprising an effective amount of a cannabinoid to improve cognitive function and ameliorate the pathophysiology of a neurodegenerative disease in a subject in need thereof.

12. The pharmaceutical composition of claim 11, wherein the composition reduces or inhibits the β-amyloid formation and aggregation in brain tissues of a subject having the neurodegenerative disease.

13. The pharmaceutical composition of claim 11, wherein the composition activates glial cell function to stimulate neuro-protective factors IL-33 and TREM2.

14. The pharmaceutical composition of claim 11, wherein the composition in the subject reduces the level of inflammatory cytokines compared to cytokine levels prior to administration of cannabidiol.

15. The pharmaceutical composition of claim 14, wherein the inflammatory cytokines are selected from the group consisting of IL-6, IFNγ and TNFα and combinations thereof.

16. The pharmaceutical composition of claim 11, wherein the neurodegenerative disease is Alzheimer's disease.

17. The pharmaceutical composition of claim 11, wherein the composition is formulated for nasal administration.

18. The pharmaceutical composition of claim 11, wherein the cannabinoid is selected from the group consisting of cannabidiol (CBD), tetrahydrocannabinols (THC), delta-9-tetrahydrocannabinol and delta-8-tetrahydrocannabinol, cannabinol (CBN), tetrahydrocannabivarin (THCV), cannabigerol (CBG), cannabidivarin (CBDV) and cannabichromene (CBC), cannabicyclol (CBL), cannabichromevarin (CBCV), cannabigerovarin (CBGV), cannabigerol monomethyl ether (CBGM), arachidonoylethanolamine (AEA), 2-arachidonoylglycerol (2-AG), 2-arachidonyl glyceryl ether (noladin ether), N-arachidonoyl dopamine (NADA), virodhamine (OAE) lysophosphatidylinositol (LPI), nabilone, rimonabant, JWH-073, CP-55940, dimethylheptylpyran, HU-210, HU-331, SR144528, WIN 55,212-2, JWH-133, levonantradol, and AM-2201 and combinations thereof.

19. The pharmaceutical composition of claim 11, wherein the cannabinoid is administered in combination with another medicament.

20. The pharmaceutical composition of claim 11, wherein the cannabinoid is administered in combination with a pharmaceutically acceptable excipient.
